(19) United States
(12) Patent Application Publication
(10) Pub. No.: US 2016/0263316 A1
(43) Pub. Date: Sep. 15, 2016

(54) METHODS AND SYSTEMS FOR COMMUNICATING WITH AN INSULIN ADMINISTERING DEVICE

(71) Applicant: Glucome Ltd., Yarkona (IL)

(72) Inventors: Dov Moran, Kfar Saba (IL); Yiftah Ben Aharon, Bakoa (IL); Menahem Lasser, Kohav Yair (IL)

(21) Appl. No.: 15/068,597

(22) Filed: Mar. 13, 2016

Related U.S. Application Data
(60) Provisional application No. 62/131,863, filed on Mar. 12, 2015.

(51) Int. Cl. A61M 5/172

(52) U.S. Cl. CPC A61M 5/172; A61M 2205/3569; A61M 2205/502

(57) ABSTRACT

A system, a device and a method associated with an insulin administering device is provided to control administering and recording of information associated with the administering. The insulin administering device is configured to connect with a computing device, such as a mobile phone, a tablet PC, a wearable device, a laptop computer or other suitable device, communicating commands and status updates. The control of such administering system may be useful for medical assessment and care of a subject suffering from a medical condition requiring administering of injected medicines such as a diabetic patient with need to inject insulin or glucagon regularly. The current invention may use various administering devices such as an insulin pen, a syringe, an insulin pump and more. The communication channel between the computing device and the insulin administering device may use audio communication channel, visual communication channel or capacitive communication channel.

### CROSS-REFERENCE TO RELATED APPLICATIONS

This application claims the benefit of priority from U.S. Provisional Patent Application No. 62/131,863, filed Mar. 12, 2015, the contents of which are incorporated by reference in their entirety.

### FIELD OF THE INVENTION

The disclosure herein relates to systems, devices and methods for communicating messages between an insulin administering device and a separate computing device. In particular, the disclosure relates to using a computing device, such as a wearable device, a mobile phone or a tablet PC in order to obtain data from such an insulin administering device or to send commands to such a device.

### BACKGROUND OF THE INVENTION

Diabetes is a metabolic disease characterized by high blood sugar, or glucose, resulting from disruption in production of, or lack of proper response to, insulin, a hormone central to regulating carbohydrate and fat metabolism.

More than 18.2 million people (or 6.3 percent of the population) in the United States suffer from diabetes. Diabetes can cause serious health complications including heart disease, blindness, kidney failure, and lower-extremity amputations. These complications may be avoided through effective and efficient balance of sugar levels. Insulin is a necessary part of a treatment plan for all people with Type 1 diabetes and many with Type 2. Insulin helps getting glucose from the bloodstream into the muscle and fat cells and needs to be injected or infused into the fatty tissues. There are various insulin administering devices such as injection devices operable to inject an insulin dose to the subject's body, including syringes, insulin pens, jet injectors and insulin pumps. The injection may be solicited, upon subject's request, or unsolicited, which may involve an automatically triggered action, according to a preconfigured setting or a command sent remotely to the insulin administering device by another device.

Examples of insulin administering devices include the syringe, which is the most common method for insulin delivery and may be a simple, disposable pump consisting of a plunger fitting tightly in a barrel and a fine coated syringe needle attached. Another example is the injection pen which may include a cartridge to contain the insulin, a needle to deliver the insulin dose, and a dial or other control buttons used to measure the dose. Still another example is an electro-mechanical pump, which is a device with a controller, a solution reservoir and an infusion set.

Accordingly and as appropriate, for insulin administration, a subject may use a syringe device for injecting insulin, an insulin pen such as Symlin pen, SoloStar, NovoPen and the like. Alternatively, another type of an insulin pump may be used such as Animas, Accucheck, Insulet onmipod and the like. Thus, a subject may set the required dosage of liquid for the injection and inject. Commonly the subject may need to record his actions prior to, during and after the injection. Recording may include the injected dosage, date and time of injections, medical condition and more.

The process, as described hereinabove, is rather cumbersome, requiring the subject to perform manually the loading of the insulin injection device with the required dosage; and logging the consumed dosage and associated information. It is noted that most insulin injection devices are disposables, with a relatively low cost of material. Any associated computerized solution using advanced communication methods such as Near Field Communication (NFC), Bluetooth (BT/BLE), Wireless Fidelity (Wi-Fi) or cellular connectivity may require costly electronic components, making the integration of such devices to many of the insulin injection devices not feasible. Therefore, there is a need to provide a simplified way for performing insulin injection and recording necessary information based upon a viable low cost communication solution.

### SUMMARY OF THE INVENTION

It is an advantage of the current disclosure that it may improve medical monitoring and injection usage by enabling a subject to control and/or to log their insulin injection device using an associated communication device. The system described herein may provide a low cost reliable and easy-to-use system managing such process.

Aspects of the disclosure present a communication device (such as a wearable device, a mobile phone, a tablet PC etc.) that connects to an insulin injection device. The communication device may send commands to the insulin injection device related to the required dosage or time of injection. It may receive from the insulin injection device information regarding the dosage that was injected, as well as some meta information (e.g. time and date or patient's medical condition).

The system may include: at least one insulin administering device; at least one computing device (such as a wearable device/mobile phone/tablet PC etc.), at least one pair of input-output mechanisms selected from the following list:
Audio (e.g. a microphone and an audio source such as a speaker/buzzer)
Visual (e.g. a camera and a light source such as a display/LED)
Capacitive (e.g. a capacitive touch screen and a capacitance source such as a capacitor or the human body).

Each one of the three input-output mechanisms listed above can be used in order to establish a low cost one-way or two-way communication channel between the insulin injection device and the communication device. The audio mechanism may utilize the audio source in order to transfer data modulated on sound waves. The visual mechanism may utilize the camera and the source of light in order to transfer data modulated on the light beams. The capacitive mechanism may utilize the capacitive touch screen and the source of capacitance in order to establish a touch-based communication, generating touch events on the touch screen by controlling the capacity of the source of capacitance

It may be noted that in order to implement the methods or systems of the disclosure, various tasks may be performed or completed manually, automatically, or combinations thereof. Moreover, according to selected instrumentation and equipment of particular embodiments of the methods or systems of the disclosure, some tasks may be implemented by hardware, software, firmware or combinations thereof using an operating system. For example, hardware may be implemented as a chip or a circuit such as an ASIC, integrated circuit or the like. As software, selected tasks according to embodiments of the disclosure may be implemented as a plurality of software instructions being executed by a computing device using any suitable operating system.

In various embodiments of the disclosure, one or more tasks as described herein may be performed by a data processor, such as a computing platform or distributed computing system for executing a plurality of instructions. Optionally, the data processor includes or accesses a volatile memory for storing instructions, data or the like. Additionally or alternatively, the data processor may access a non-volatile storage, for example, a magnetic hard-disk, flash-drive, removable media or the like, for storing instructions and/or data. Optionally, a network connection may additionally or alternatively be provided. User interface devices may be provided such as visual displays, audio output devices, tactile outputs and the like. Furthermore, as required user input devices may be provided such as keyboards, cameras, microphones, accelerometers, motion detectors or pointing devices such as mice, roller balls, touch pads, touch sensitive screens or the like.

It is according to one aspect of the disclosure to teach a method for administering an insulin dose to a subject comprising: administering, by an insulin administering device, an insulin dose to the subject; generating, by the insulin administering device, at least one message containing a status update; and sending, by the insulin administering device, the at least one message to a computing device via a communication channel, where the computing device being separate from the insulin administering device, wherein the communication channel is one of an audio communication channel, a visual communication channel, and a capacitive communication channel.

Variously, the status update comprises at least one of: an administer success notification, an administer failure notification, an administered dosage notification; a fluid remaining notification, a battery condition notification, a next scheduled injection notification.

Optionally, in accordance with a selected embodiment of the present disclosure, the communication channel is an audio communication channel.

Optionally, in accordance with another selected embodiment of the present disclosure, the communication channel is a visual communication channel.

Optionally, in accordance with yet another selected embodiment of the present disclosure, the communication channel is a capacitive communication channel.

Additionally, the method may further comprise the step of receiving, by the computing device, a status update from the insulin administering device.

Additionally, the method may further comprise the step of receiving a command, by the insulin administering device, from the computing device via the communication channel, the command instructing the insulin administering device to carry out the administering.

Optionally, in accordance with a selected embodiment of the present disclosure, the command includes an insulin dosage to be used in the administering.

As appropriate, the generating and the sending steps are in response to the administering.

According to another aspect of the disclosure, in accordance with a selected embodiment of the present disclosure, a system for administering an insulin dose to a subject is disclosed, the system comprising: an insulin administering device operable to administer the insulin dose and to generate at least one message containing a status update; a computing device separate from said insulin administering device; and a communication channel operable to communicate the at least one message from said insulin administering device to the computing device, wherein the communication channel is selected from a group consisting of: an audio communication channel, a visual communication channel, and a capacitive communication channel.

Optionally, in accordance with a selected embodiment of the present disclosure, the computing device comprises a microphone operable to detect the at least one message.

In yet another aspect of the disclosure, in accordance with a selected embodiment of the present disclosure, an insulin administering device is disclosed which is operable to administer an insulin dose, the insulin administering device comprising: a processor operable to generate at least one message containing a status update; and a signal transmitter operable to communicate the at least one message to a computing device separate from the insulin administering device via a communication channel, wherein the communication channel is selected from a group consisting of: an audio communication channel, a visual communication channel, and a capacitive communication channel.

Optionally, in accordance with a selected embodiment of the present disclosure, the communication channel is an audio communication channel. As appropriate, the insulin administering device comprises a processor operable to encode the at least one message into a sound wave and an audio emitter operable to emit the sound wave. Optionally, the computing device comprises a microphone operable to detect the sound wave.

Optionally, in accordance with a selected embodiment of the present disclosure, the communication channel is a visual communication channel. As appropriate, the insulin administering device comprises a processor operable to encode said at least one message into a visually-encoded representation and a visual display for presenting the visually-encoded representation. Optionally, the visually-encoded representation comprises a geometric pattern.

Variously, the visually-encoded representation is selected from a group including a one-dimensional barcode, a two-dimensional barcode and alphanumeric characters. Furthermore, the visually encoded representation comprises a time sequence of visual effects.

Optionally, and where appropriate, the visual display comprises one or more LEDs operable to flash according to instructions from the processor of the insulin administering device, thereby encoding the at least one message. Additionally or alternatively, the visual display comprises one or more multi-color LEDs, operable to generate colors according to instructions from the processor of the insulin administering device, thereby encoding the at least one message.

Optionally, in accordance with a selected embodiment of the present disclosure, the communication channel is a capacitive communication channel. Accordingly, the insulin administering device comprises a processor operable to encode the at least one message into a capacitive profile and at least one capacitive profile output mechanism operable to represent the capacitive profile. Additionally or alternatively, the capacitive profile comprises a sequence of capacitive states varying over a period of time. Furthermore, the capacitive profile may comprise a plurality of regions, each exhibiting a capacitive state. Additionally, each region of the plurality of regions may exhibit a sequence of capacitive states varying over a period of time.

### BRIEF DESCRIPTION OF THE DRAWINGS

For a better understanding of the embodiments and to show how it may be carried into effect, reference will now be made, purely by way of example, to the accompanying drawings.

With specific reference now to the drawings in detail, it is stressed that the particulars shown are by way of example and for purposes of illustrative discussion of selected embodiments only, and are presented in the cause of providing what is believed to be the most useful and readily understood description of the principles and conceptual aspects. In this regard, no attempt is made to show structural details in more detail than is necessary for a fundamental understanding; the description taken with the drawings making apparent to those skilled in the art how the several selected embodiments may be put into practice. In the accompanying drawings:

FIG. 1A is a schematic illustration of a control system of an insulin administering device according to one embodiment of the presently disclosed subject matter;

FIG. 1B is a schematic block diagram of an insulin administering device according to one embodiment of the presently disclosed subject matter;

FIGS. 1C and 1D schematically show the top side and the underside respectively of a possible insulin administering device for use with a system incorporating a capacitive communication channel;

FIG. 2 is a schematic illustration of a networked control system of an insulin administering device according to another embodiment of the presently disclosed subject matter;

FIG. 3 is a flowchart illustrating one method for controlling the communication between the computing device and the insulin administering device according to the presently disclosed subject matter;

FIG. 4 is a flowchart illustrating a method for communicating a status update between the insulin administering device and the computing device according to the presently disclosed subject matter; and

FIG. 5 is a block diagram illustrating an example of a possible structure of a command message representing the various command fields.

### DETAILED DESCRIPTION OF THE INVENTION

It is noted that the systems and methods of the invention herein, may not be limited in their application to the details of construction and the arrangement of the components or methods set forth in the following description or illustrated in the drawings and examples. The systems and methods of the invention may be capable of other embodiments or of being practiced or carried out in various ways.

Alternative methods and materials similar or equivalent to those described herein may be used in the practice or testing of embodiments of the disclosure. Nevertheless, particular methods and materials are described herein for illustrative purposes only. The materials, methods, and examples are not intended to be necessarily limiting.

Accordingly, various embodiments may omit, substitute, or add various procedures or components as appropriate. For instance, it should be appreciated that the methods may be performed in an order different than described, and that various steps may be added, omitted or combined. Also, aspects and components described with respect to certain embodiments may be combined in various other embodiments. It should also be appreciated that the systems, methods, devices, and software may individually or collectively be components of a larger system, wherein other procedures may take precedence over or otherwise modify their application.

The disclosure herein relates to systems and methods for controlling an insulin administering device. In particular, the disclosure relates to using a computing device, such as a communicator device, wearable device, a mobile phone, a tablet, a laptop and the like in order to control and to receive updates from such an insulin administering device, which may be useful for medical assessment and care of a subject suffering from a medical condition requiring administering of injected medicines, for example, a diabetic patient with need to inject insulin or glucagon regularly.

By way of illustration, the current invention may use various administering devices and various communication channels.

In one embodiment of the invention, an insulin pen having an audio signal transmitter may communicate audio signals to a mobile device having a microphone, operable to detect the message signals.

Another embodiment of the invention may use an insulin pump having a source of light, a display or other visual communication, connecting to a mobile device having an image capture device such as a camera operable to detect the visually communicated messages.

Yet, another embodiment of the invention may use a syringe having a capacitive output mechanism connecting to a mobile device having a capacitive touch screen.

Still other embodiments may involve other combinations of communication channels and administering devices.

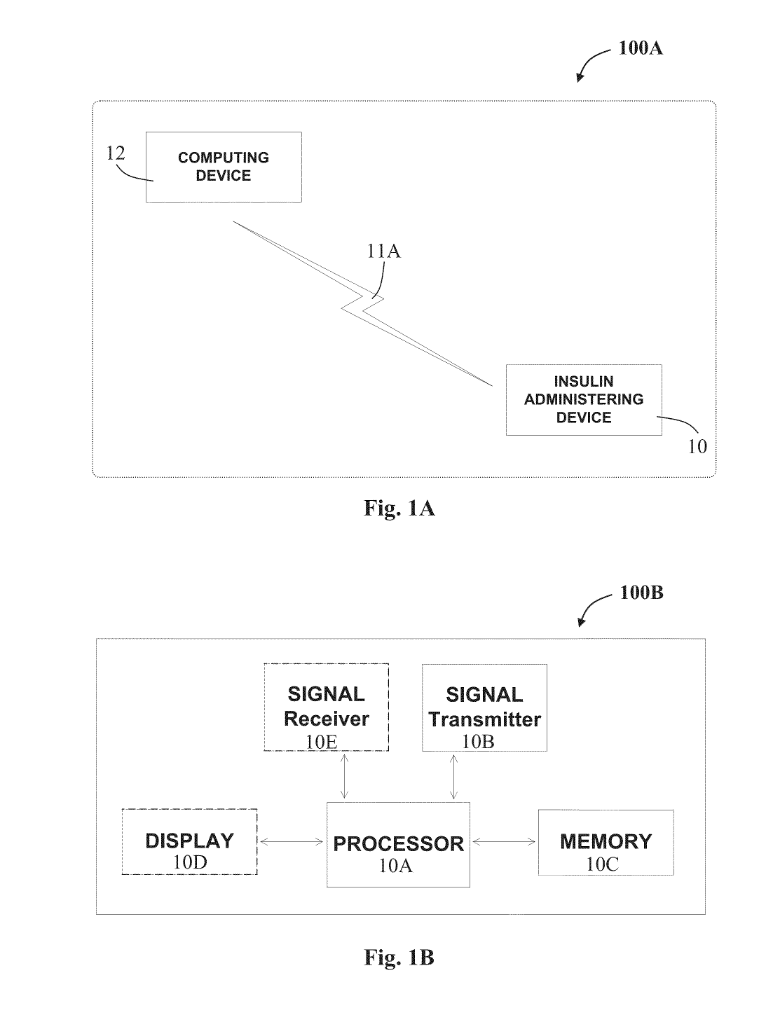

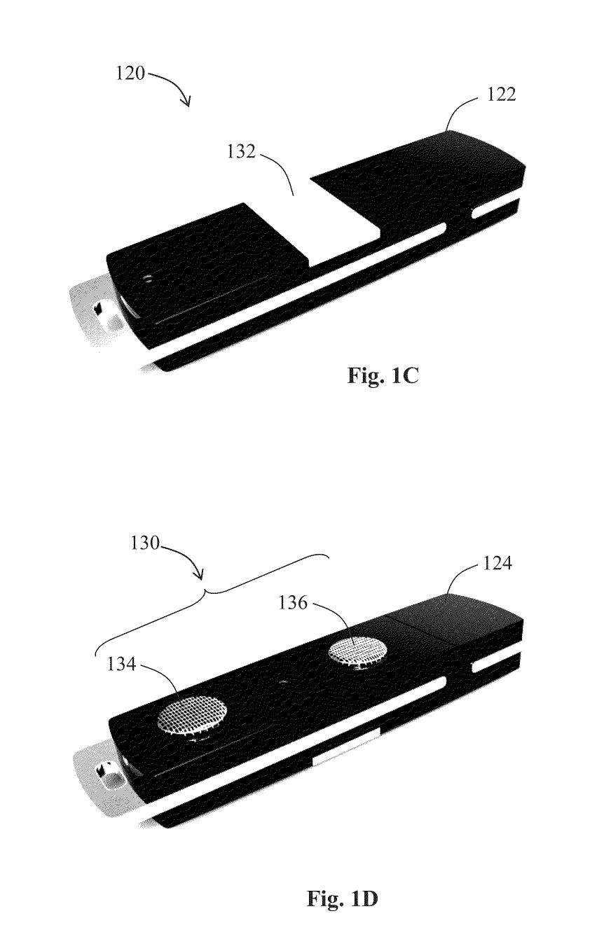

As illustrated in FIG. 1A, there is provided a control system for an insulin administering device, which is generally indicated at 100A, for controlling the insulin administering to enable proper injection and recording the information associated with the action of injection.

The control system 100A comprises at least one insulin administering device 10 operable to administer an insulin dose and generate at least one message containing a status update. The insulin administering device 10 is further operable to connect with at least one computing device 12 such as a mobile phone, a tablet, a wearable device, a laptop and the like via a communication channel 11A.

The computing device 12, comprising a processor (not shown), is separate from the administering device 10. Accordingly, the computing device 12 may control the insulin administering device 10 by sending command messages related to the required dosage, time of injection and the like. Additionally, the computing device 12 is operable to receive from the insulin administering device 10 at least one message comprising information and status updates associated with the administered dose as well as meta information such as time and date, a subject's medical condition and more.

The communication channel 11A is operable to communicate both the command messages from the computing device 12 to the insulin administering device 10 and the at least one message from the insulin administering device 10 to the computing device 12, and may be selected from a group consisting of: an audio communication channel, a visual communication channel, a capacitive communication channel and the like, as well as combinations thereof.

As illustrated in FIG. 1B, there is provided a block diagram of an insulin administering device, which is generally indicated at 100B, for performing the insulin administering, recording the associated data and communicating with a remote computing device.

The block diagram of an insulin administering device 100B comprises a processor 10A operable to generate at least one message containing a status update associated with an administering operation; a signal transmitter 10B operable to communicate the at least one message to a computing device separate from the insulin administering device via a communication channel (FIG. 1A, item 11A) and a memory unit 10C configured to store status updates related information.

Optionally, the processor 10A associated with the insulin administering device, is operable to execute commands received from the computing device (FIG. 1A, item 12) via a signal receiver 10E.

It is noted that the signal receiver 10E is only required if two-way communication is desired. Optionally, the signal receiver 10E is an audio receiver.

Optionally, the processor 10A associated with the insulin administering device, is operable to encode at least one message into a sound wave and to activate an audio emitter, where the audio emitter may be a speaker, a buzzer and the like, operable to emit the sound wave (not shown).

Optionally, the processor 10A associated with the insulin administering device, is operable to encode at least one message into a visually-encoded representation and use a visual display 10D for presenting the visually-encoded representation. Furthermore, the processor 10A is operable generate a time sequence of visual effects. The sequence of visual effects may be generated by one or more LEDs operable to flash in a readable manner thereby communicating at least one message.

Optionally, the processor 10A associated with the insulin administering device, is operable to encode at least one message into a capacitive profile and use at least one capacitive profile output mechanism operable to represent the capacitive profile (not shown).

It is noted that the signal transmitter 10B is operable to communicate the message signals according to the type of communication channel (FIG. 1A, item 11A) used—audio communication channel, visual communication channel or capacitive communication channel, as described hereinafter. Further, each of the three input-output mechanisms listed above may be operable to establish a low cost one-way or two-way communication channel between the insulin administering device (FIG. 1A, item 10), and the computing device (FIG. 1A, item 12).

### Audio Communication:

The audio communication channel may utilize the audio source in order to transfer data modulated on sound waves. Specifically, the insulin administering device is operable to transmit at least one message encoded into a sound wave generated by the speaker/buzzer associated with the insulin administering device and further detectable by a microphone associated with the computing device (FIG. 1A, item 12), for one-way communication channel. Two-way communication channel may require a signal receiver associated with the insulin administering device, as described in FIG. 1B, hereinabove.

By way of example, an audio emitter such as a speaker may be configured to emit at least two distinct sounds, distinguishable by at least one of pitch, frequency, amplitude, timbre, duration, characteristic pulse frequency or the like. Accordingly, a first distinct sound may be designated to represent logic state ZERO and a second distinct sound may be designated to represent logic state ONE. Each emitted sound may thus represent a bit of data and a string of eight such bits may represent a full byte of data as required.

As appropriate, a detailed example and description of an audio-based input-output mechanism is disclosed in U.S. application Ser. No. 14/071,744 titled “Method for Collecting Medical Data and Associated System”, the contents of which are incorporated by reference in their entirety.

### Visual Communication:

The visual communication channel may use a camera or any kind of light sensor such as photoelectric cell, IR sensors, photodetector and the like, associated with the computing device 12 operable to detect a light source activated by the insulin administering device (FIG. 1A, item 10), for example using a visual display/LED. Specifically, the visual mechanism may transfer data modulated on the light beams between the insulin administering device and the computing device.

The visual display (FIG. 1B, item 10.4), associated with the insulin administering device (FIG. 1A, item 10) may comprise one or more elements configured to visually present encoded data of a communication message. The visual display may comprise one or more LEDs such as multi-color LEDs being configured to selectively produce light of different colors, a screen, such as LCD, LED, OLED, plasma display, ELD, electronic paper, or electronic ink, or any other suitable display elements. In addition, it may comprise a combination of two or more different display technologies. Further, the computing device (FIG. 1A, item 12) may use a processor configured to facilitate displaying of decoded information of the message. The encoding and decoding may be accomplished by any method known in the art. The method of displaying the encoded data on the visual display of the insulin administering device is dependent on the type of visual display.

By way of example, a visually encoded communication message may be encoded as a sequence of visual elements. Where appropriate, the visual display may be configured to emit at least two distinct visual states, distinguishable by at least one of spatial position, color, on/off state, frequency, wavelength, brightness, amplitude, duration, flash rate, characteristic pulse profile, or the like as well as combinations thereof. Accordingly, a first visual state may be designated to represent logic state ZERO and a second visual state may be designated to represent logic state ONE. Each displayed element may thus represent a bit of data and a string of eight such bits may represent a full byte of data as required.

For the sake of illustrative example only, according to a modification wherein the visual display (FIG. 1B, item 10D) comprises one or more LEDs, the information may be encoded and displayed as a time sequence of on/off states of the LEDs. In addition, different colors may be used to encode values of data. (For example, a multi-color LED may encode two bits of binary data in a single flash thereof, wherein each of four different colors indicates one of 00, 01, 10, and 11.) In addition, different durations of a flash may indicate different values. Combinations of the above may be employed, wherein the value transmitted by an LED depends both on the color and duration of its flash. According to some variations, the visual display (FIG. 1B, item 10D) comprises several LEDs, wherein the processor (FIG. 1B, item 10A) is configured to transmit data separately via each LED simultaneously, jointly using all LEDS as a single data channel, or in combinations thereof.

The communication message may be encoded as a pattern. For example, according to a modification wherein the visual display (FIG. 1B, item 10D) comprises a screen, the processor (FIG. 1B, item 10A) is configured to encode the information and direct the visual display (FIG. 1B, item 10D) to present it as alphanumeric characters. According to other variation, the processor (FIG. 1B, item 10A) is configured to encode the communication message and direct the visual display to present it as a barcode, e.g., as a one-dimensional barcode or a two-dimensional barcode. It will be appreciated that if the visual display is a color display, information may be encoded using different colors to indicate different values of encoded data.

As appropriate, a detailed example and description of a visual-based input-output mechanism is disclosed in U.S. application Ser. No. 14/133,706 titled “Methods and Systems for Analyzing a Blood Sample”, the content of which are incorporated by reference in their entirety.

In addition to information relating to administering, the visual display (FIG. 1B, item 10D) may present error-detection and/or error-correction information, e.g., as is well-known in the art.

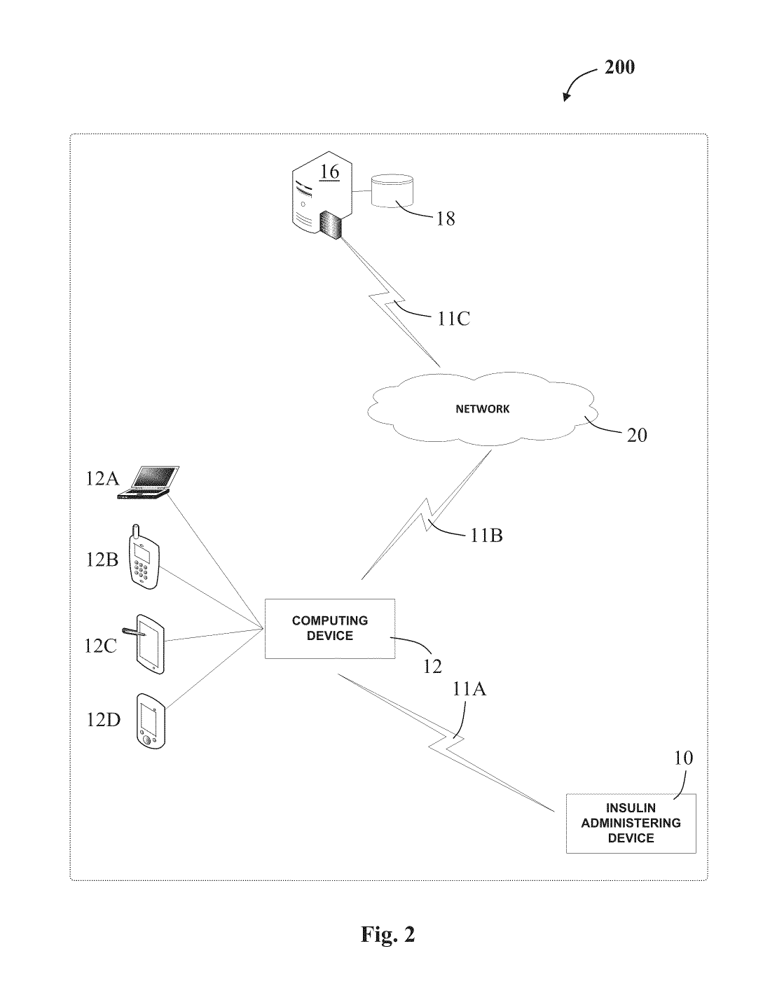

The computing device (FIG. 1A, item 12) is any suitable device such as a wearable device, a mobile device, a smartphone, a tablet computer or any other suitable device configured to receive information transmitted by the visual display of the insulin administering device (FIG. 1A, item 10), execute a program, display information to a user, and optionally receive commands from a user. It may also be configured to communicate with an external network, for example a public network such as the Internet, a POTS network, an ISDN network, cellular telephone system, and/or a VoIP system (see FIG. 2). In particular, the computing device (FIG. 1A, item 12) is configured for installation thereon of third-party software.

Capacitive Communication:

The capacitive communication channel may use a capacitive touch screen associated with the computing device (FIG. 1A, item 12) and a capacitive output mechanism associated with insulin administering device (FIG. 1A, item 10). The capacitive mechanism may utilize the capacitive touch screen and the capacitive output mechanism may use a capacitance source such as a capacitor or the human body in order to establish a touch-based communication, generating touch events on the touch screen by controlling the capacity of the source of capacitance. The capacitive output mechanism may be configured to produce a capacitive profile, i.e., a pattern of capacitive states. The capacitive profile may be time-based, wherein the capacitive output mechanism exhibits a sequence of varying capacitive states (i.e., levels of electrical charge storage capacity) over a period of time, for example changing between exhibiting no electrical charge storage capacity and a non-zero value of electrical charge storage capacity. Alternatively, as will be described below, the capacitive profile may be location-based. In addition, the capacitive profile may be a combination of location-based and time-based. According to any example, the capacitive profile may include error-detection and/or error-correction information, e.g., as is well-known in the art.

For example, the capacitive output mechanism may use a surface defined by nine regions which is configured for producing a location-based capacitive profile. Each region being electrically connected on a back side thereof to a switch, which is configured to selectively toggle its respective region between connected and disconnected states with electrical charged conductance source. The source may be electronics-based. Alternatively, it may be a surface of a display (FIG. 1B, item 10D) associated with the insulin administering device (FIG. 1A, item 10) which is positioned so as to be in contact with a user's hand while in use, thereby taking advantage of the natural electrical charge conductance of the user. The processor (FIG. 1B, item 10A) may be configured to control each of the switches such that its respective region displays the proper capacitive state (i.e., electrical charge storage capacity), e.g., at the proper time.

As appropriate, a detailed example and description of a capacitance-based input-output mechanism is disclosed in U.S. application Ser. No. 14/133,706, already incorporated by reference above.

As mentioned, the processor (FIG. 1B, item 10A) is configured to facilitate representation of encoded information regarding the status update via the capacitive output mechanism. This representation is accomplished by controlling the capacitive profile.

According to variations wherein the capacitive profile is time-based, the duration of time for which the capacitive output mechanism exhibits, e.g., a non-zero value of electrical charge storage capacity, may represent a certain value. For example, a predetermined interval of non-zero electrical charge storage capacity may represent the binary digit ONE, while the same interval of no electrical charge storage capacity may represent the binary digit ZERO.

According to variations wherein the capacitive profile is location-based, each region may represent a bit in a binary string. One or more of the regions may be utilized to indicate the orientation of the surface, for example by rapidly toggling its capacitive state in a predetermined fashion.

Where appropriate, the capacitive output mechanism may be configured to produce two distinct capacitive states, distinguishable by at least one of spatial position, on/off state, voltage, amplitude, duration, characteristic pulse profile, or the like as well as combinations thereof. Accordingly, a first capacitive state may be designated to represent logic state ZERO and a second capacitive state may be designated to represent logic state ONE. Each displayed element may thus represent a bit of data and a string of eight such bits may represent a full byte of data as required.

According to variations wherein the capacitive profile is a combination of location-based and time-based, each region may produce a time-based capacitive profile independent of the other regions. In this way, multiple time-based capacitive profiles may be produced simultaneously, increasing the rate at which encoded information is represented via the capacitive output mechanism. One or more of the regions may be utilized to indicate the orientation of the surface, for example by rapidly toggling its capacitive state in a predetermined fashion.

Referring now to FIGS. 1C and 1D which show a top and an underside view of possible embodiments of an insulin administering device 120 incorporating capacitive output mechanisms 130. The top side 122 of the insulin administering device 120 includes a touch pad 132 and the underside 124 of the insulin administering device 120 includes two distinct output contacts 134, 136. In order to communicate a capacitive signal to a computing device, the output contacts 134, 136 may both be pressed against a capacitive input mechanism of a computing device, such as the touchscreen of a mobile telephone or the like. The user may then place a finger upon the touch pad 132 upon the top side 122 of the insulin administering device 120. Internal circuitry (not shown) may selectively connect either the first output contact 134 or the second output contact 136 to the touch pad 132.

In still other embodiments, the internal circuitry of the insulin administering device may include an internal capacitor which may be selectively connected to either the first output contact or the second output contact such that there may be no need for the touch pad.

The internal circuitry typically includes a first electronic switch, a second electronic switch and a microcontroller. The first electronic switch, such as a transistor, a micro switch, a MOSFET, a Relay or the like, is configured to conductively connect the first output contact 134 to a capacitive source such as the touch pad 132 or an internal capacitor. The second electronic switch is configured to conductively connect the second output contact 136 to a capacitive source such as the touch pad 132 or an internal capacitor. The microcontroller may be wired to the gate terminals of the electronic switches or be otherwise operable to provide control signals to selectively trigger one or both of the first or the second electronic switches.

When the first output contact 134 is connected to the touch pad 132, the capacitive input mechanism may detect a change in the capacitive state at the first output contact 134 which may indicate a logical state of ZERO for example. Similarly, when the second output contact 136 is connected to the touch pad 132, the capacitive input mechanism may detect a change in the capacitive state at second output contact 136 which may indicate a logical state of ONE for example. Thereby bits of data may be communicated to the computing device.

Additionally, the computing device (FIG. 1A, item 12) such as a smartphone and the like may include a processor, one or more memory modules (which may comprise volatile and/or non-volatile memory), a capacitive sensing user-input interface, a user-output interface, and a power source. The capacitive user-input interface and user-output interface may be part of the same element, e.g., a capacitive touch-screen may constitute both. According to some modifications, the capacitive user-input interface may use multi-touch technology, i.e., it is configured to detect capacitive input at several locations simultaneously.

In addition, the computing device (FIG. 1A, item 12) may comprise a transceiver, such as a modem and/or a wireless network adapter, configured to communicate with the external network (FIG. 2, item 20).

The processor associated with the computing device (FIG. 1A, item 12), may be configured to direct operation of the computing device (FIG. 1A, item 12). Inter alia, it is configured to execute software stored in the memory. In addition, the processor may be configured to facilitate updating software stored in the memory, for example by downloading updated software from a remote server via the Internet.

In particular, the processor associated with the computing device (FIG. 1A, item 12) is configured to analyze a capacitive profile captured by the capacitive user-input interface. It is configured to analyze the detected capacitive profile to establish whether it contains encoded data, and to decode the data. The computing device may be loaded with a software application which is configured to facilitate the decoding. For example, the information transmitted by the insulin administrator device (FIG. 1A, item 10) may contain raw data, which the software application is configured to interpret and provide a useful value based thereon.

It is noted that each one of the input-output mechanisms described hereinabove may be used in order to establish a low cost one-way or two-way communication channel (FIG. 1A, item 11A) between the insulin administering device (FIG. 1A, item 10) and the computing device (FIG. 1A, item 12).

It is further noted that in order to implement the methods or systems of the disclosure, various tasks may be performed or completed manually, automatically or combinations thereof.

As illustrated in FIG. 2, there is provided a networked control system of an insulin administering device, which is generally indicated at 200, for controlling the insulin administering to enable proper injection, recording the information associated with the action of injection and storing in a system repository to enable keeping a medical profile of a subject.

The networked control system 200 comprises at least one insulin administering device 10 operable to administer an insulin dose and generate at least one message containing a status update. The insulin administering device 10 is further operable to connect with at least one computing device 12 via a communication channel 11A. The computing device 12 is operable to communicate via a communication network 20 with a remote central server 16 associated with a system repository 18 configured to store associated medical records.

It is noted that communication with the remote central server 16 via the external network 20 may use for example a public network such as the Internet, a POTS network, an ISDN network, cellular telephone system, and/or a VoIP system.

It is further noted that the computing device 12 may be selected from various portable devices, such as a laptop computer 12A, a mobile phone (smartphone) 12B, a tablet computer 12C, a wearable device 12D and any other suitable device.

It is further noted that according to selected instrumentation and equipment of particular embodiments of the methods or systems of the disclosure, some tasks may be implemented by hardware, software, firmware or combinations thereof using an operating system. For example, hardware may be implemented as a chip or a circuit such as an ASIC, integrated circuit or the like. As software, selected tasks according to embodiments of the disclosure may be implemented as a plurality of software instructions being executed by a computing device using any suitable operating system.

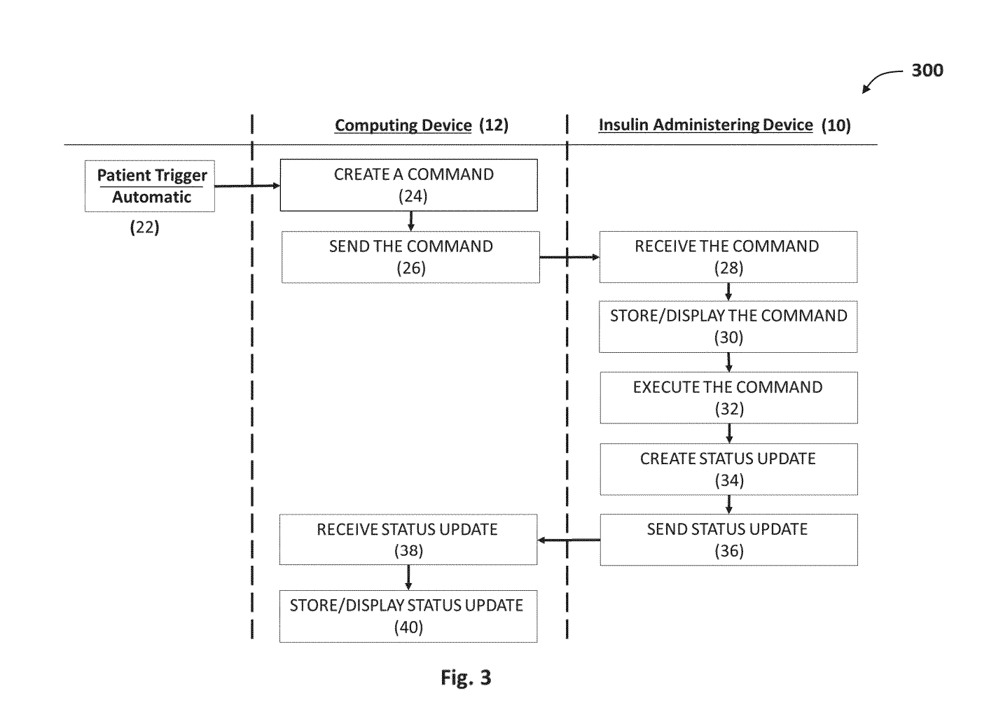

As illustrated in FIG. 3, there is provided a flowchart illustrating a method, which is generally indicated at 300, for controlling a communication process between the computing device (FIG. 1A, item 12) and the insulin administering device (FIG. 1A, item 10).

The controlling process may start by the subject instructing the computing device (FIG. 1A, item 12) to start an injection activity—step (22). Alternatively, the injection activity may start automatically, without an explicit subject intervention. The automatic triggering may be performed according to a schedule of a pre-set starting time, or according to a pre-defined condition such as when the insulin level of the subject gets to a pre-defined level.

Thereafter, creating a command associated with the injection activity,—step 24, by the computing device (FIG. 1A, item 12), instructing the insulin administering device to carry out the administering. As appropriate, the command may include various parameters such as the dosage to use for the administering; and sending the command to the insulin administering device (FIG. 1A, item 10)—step 26, via the communication channel (FIG. 1A, item 11A); receiving the command by the insulin administering device (FIG. 1A, item 10)—step 28; optionally, storing/displaying of the command—step 30; thereafter, executing the command by the insulin administering device—step 32; optionally, creating a status update associated with the execution of the command—step 34; and sending the status update to the computing device (FIG. 1A, item 12)—step 36, where the status update may include various notifications selected from a group consisting of: an administer success notification, an administer failure notification, an administered dosage notification, a fluid remaining notification, a battery condition notification, a next scheduled injection time and combinations thereof.

Further, receiving the status update—step 38, by the computing device (FIG. 1A, item 12); and optionally, storing the status update in a local storage of the computing device and/or further, optionally, transmitting the status update, by the computing device to a central data collection unit such a medical database server (FIG. 2, item 16). Optionally, displaying the status update on a local display of the computing device (FIG. 1A, item 12), providing the user with immediate feedback—step 40.

Variously, as described in FIG. 1A hereinabove, the communication between the insulin administering device (FIG. 1A, item 10) and the computing device (FIG. 1A, item 12) is performed over a communication channel (FIG. 1A, item 11A), wherein the communication channel may be an audio communication channel, a visual communication channel or a capacitive communication channel.

Accordingly, sending the status update over an audio communication channel may require the insulin administering device to encode the status update message into a sound wave profile and further generate a sound wave having such a profile. Consequently, the computing device may be operable of detecting the sound wave using an associated microphone. Furthermore, sending the status update signal over a visual communication channel may require the insulin administering device to present the status update as a machine-readable visually-encoded representation.

It is noted that the visually-encoded representation may include a pattern selected from a group consisting of one-dimensional barcodes, two-dimensional barcodes and alphanumeric characters. Optionally, the visually-encoded representation may include a sequence of visual elements, such as flashes generated by one or more LEDs associated with the insulin administering device. Further, the LEDs may be of multi-colors wherein different colors of each of the LEDs represent different values of encoded data.

Moreover, sending the status update over a capacitive communication channel may require the insulin administering device to encode the status update message into a capacitive profile representing the status update via a capacitive profile output mechanism. The capacitive profile comprises a sequence of capacitive states varying over a period of time. Alternatively, the capacitive profile may include a plurality of regions, each region exhibiting a capacitive state. Optionally, each region exhibits a sequence of capacitive states that may vary over a period of time. Further, it is noted that the capacitive profile optionally includes at least one of error-correction and error-detection information.

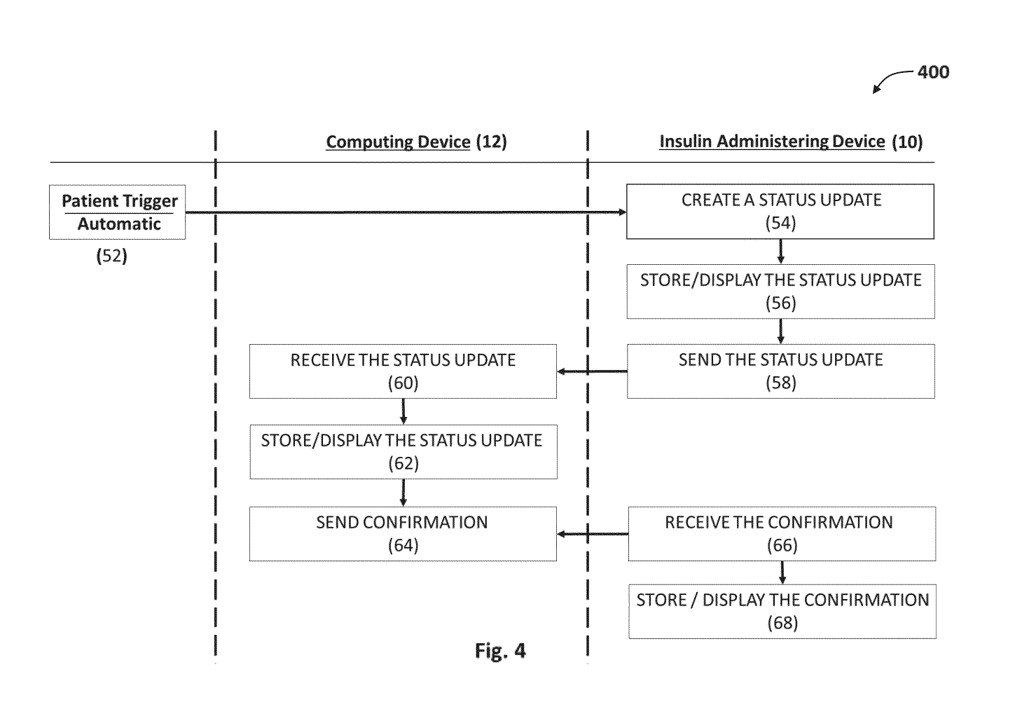

As illustrated in FIG. 4, there is provided a flowchart illustrating another method, which is generally indicated at 400, representing an example of receiving a status update from the insulin administering device (FIG. 1A, item 10) that is not associated with an injection.

The process may start by the subject, instructing the insulin administering device (FIG. 1A, item 10) to start monitoring/updating—step (52). Alternatively, the monitoring/updating activity may start automatically, without an explicit subject intervention. The automatic triggering of updates may be performed according to a schedule, a pre-set starting time for sending an update, or according to a pre-defined setting of the computing device (FIG. 1A, item 12) to request a status update.

Thereafter, creating a status update message—step 54, by the insulin administrative device (FIG. 1A, item 10); optionally, storing/displaying the status update message—step 56, on a display associated with the insulin administering device (FIG. 1A, item 10); and sending the status update message to the computing device (FIG. 1A, item 12)—step 58. Thereafter, receiving the status update message—step 60, by the computing device (FIG. 1A, item 12), where the status update message include at least one of the following notifications: an administer success notification of the last injection attempt, an administer failure notification of the last injection attempt, a administered dosage notification; a fluid remaining notification, a battery condition notification, a next scheduled injection notification; storing/displaying the status update message received, locally on the computing device or communicated remotely to a data repository—step 62; optionally, sending a confirmation message to the insulin administering device (FIG. 1A, item 10)—step 64; and receiving the confirmation message, by the insulin administering device—step 66; and where appropriate, storing the confirmation message in a memory of the insulin administering device or displaying the confirmation message on a display associated with the insulin administering device—step 68.

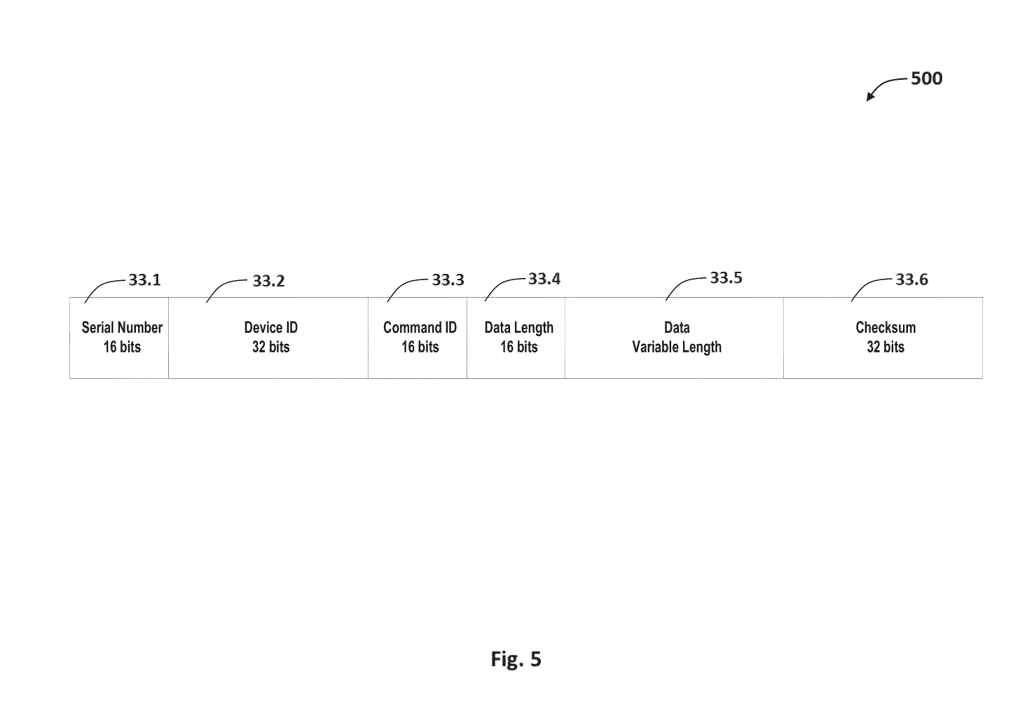

As illustrated in FIG. 5, there is provided a block diagram illustrating a possible structure of a command message or a status update message, which is generally indicated at 500, for using in managing an insulin administering device.

The block diagram of the command or status update message structure 500 comprises an exemplified possible set of fields:

(a) Serial number field of 16 bits long, incremented by one upon each message transmission.

(b) Device ID field of 32 bits long, allowing the identification of each device in the system.

(c) Command ID field of 16 bits long, representing the command/status update to be sent.

(d) Data length field of 16 bits, representing the size of additional data (payload) to be sent in multiplications of 16 bits.

(e) Data field of variable length, representing the payload to be sent.

(f) Checksum field of 32 bit, representing integrity check of the entire command.

As shown in FIG. 1A and FIG. 2, one-way or two-way communication channel may be used to implement the suggested process.

In case of a two-way communication channel, a receiver should be added to the insulin administering device.

In case of audio-based communication (for example), such a receiver may be a microphone. In this example, the computing device (FIG. 1A, item 12) may use the associated device speaker in order to generate audio-based signals, which will be received by the insulin administering device using the above mentioned microphone.

It should be appreciated that in case of two-way communication between the insulin administering device and the computing device it is not necessary that both directions of communication use the same type of channel. It is possible that commands to the insulin administering device are sent over a first communication channel of a first type (i.e. audio-based, visual-based or capacitance based) and status updates from the insulin administering device are sent over a second communication channel of a second type, which is different from the first type.

It should be appreciated to those skilled in the art that the invention may not be limited to the details of the foregoing illustrative embodiments and that the present invention may use various other embodiments in other specific forms without departing from the nature or essential attributes thereof. The present embodiments are therefore to be considered in all respects as illustrative and not restrictive.

Those skilled in the art to which this invention pertains will readily appreciate that numerous changes, variations and modifications can be made without departing from the scope of the invention mutatis mutandis.

Technical and scientific terms used herein should have the same meaning as commonly understood by one of ordinary skill in the art to which the disclosure pertains. Nevertheless, it is expected that during the life of a patent maturing from this application many relevant systems and methods will be developed. Accordingly, the scope of the terms such as computing unit, network, display, memory, server and the like are intended to include all such new technologies a priori.

The terms “comprises”, “comprising”, “includes”, “including”, “having” and their conjugates mean “including but not limited to” and indicate that the components listed are included, but not generally to the exclusion of other components. Such terms encompass the terms “consisting of” and “consisting essentially of”.

The phrase “consisting essentially of” means that the composition or method may include additional ingredients and/or steps, but only if the additional ingredients and/or steps do not materially alter the basic and novel characteristics of the composition or method.

As used herein, the singular form “a”, “an” and “the” may include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments or to exclude the incorporation of features from other embodiments.

The word “optionally” is used herein to mean “is provided in some embodiments and not provided in other embodiments”. Any particular embodiment of the disclosure may include a plurality of “optional” features unless such features conflict.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween. It should be understood, therefore, that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6 as well as non-integral intermediate values. This applies regardless of the breadth of the range.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. Certain features described in the context of various embodiments are not to be considered essential features of those embodiments, unless the embodiment is inoperative without those elements.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the disclosure.

1. A method for administering an insulin dose to a subject comprising:
administering, by an insulin administering device, an insulin dose to said subject;
generating, by said insulin administering device, at least one message containing a status update; and
sending, by the insulin administering device, said at least one message to a computing device via a communication channel, said computing device being separate from said insulin administering device, wherein said communication channel is one of an audio communication channel, a visual communication channel, and a capacitive communication channel.

2. The method of claim 1 wherein said status update comprises at least one of: an administer success notification, an administer failure notification, an administered dosage notification, a fluid remaining notification, a battery condition notification, a next scheduled injection notification.

3. The method of claim 1 wherein said communication channel is an audio communication channel.

4. The method of claim 1 wherein said communication channel is a visual communication channel.

5. The method of claim 1 wherein said communication channel is a capacitive communication channel.

6. The method of claim 1 further comprising receiving, by the computing device, a status update from the insulin administering device.

7. The method of claim 1 further comprising said insulin administering device receiving a command from said computing device via said communication channel, said command instructing said insulin administering device to carry out said administering.

8. The method of claim 7 wherein said command includes an insulin dosage to be used in said administering.

9. The method of claim 1 wherein the generating and the sending are in response to the administering.

10. A system for administering an insulin dose to a subject, the system comprising:
an insulin administering device operable to administer said insulin dose and to generate at least one message containing a status update;
a computing device separate from said insulin administering device; and
a communication channel operable to communicate said at least one message from said insulin administering device to said computing device,
wherein said communication channel is selected from a group consisting of: an audio communication channel, a visual communication channel, and a capacitive communication channel.

11. The system of claim 10 wherein the computing device comprises a microphone operable to detect said at least one message.

12. An insulin administering device operable to administer an insulin dose, said insulin administering device comprising:
a processor operable to generate at least one message containing a status update; and
a signal transmitter operable to communicate said at least one message to a computing device separate from said insulin administering device via a communication channel,
wherein said communication channel is selected from a group consisting of: an audio communication channel, a visual communication channel, and a capacitive communication channel.

13. The device of claim 12 wherein said communication channel is an audio communication channel.

14. The device of claim 13 wherein the insulin administering device comprises a processor operable to encode said at least one message into a sound wave and an audio emitter operable to emit the sound wave.

15. The device of claim 14 wherein the computing device comprises a microphone operable to detect said sound wave.

16. The device of claim 12 wherein said communication channel is a visual communication channel.

17. The device of claim 16 wherein the insulin administering device comprises a processor operable to encode said at least one message into a visually-encoded representation and a visual display for presenting the visually-encoded representation.

18. The device of claim 17 wherein the visually-encoded representation comprises a geometric pattern.

19. The device of claim 17 wherein the visually-encoded representation is selected from a group including a one-dimensional barcode, a two-dimensional barcode and alphanumeric characters.

20. The device of claim 17 wherein the visually encoded representation comprises a time sequence of visual effects.

21. The device of claim 17 wherein the visual display comprises one or more LEDs operable to flash according to instructions from the processor of the insulin administering device, thereby encoding said at least one message.

22. The device of claim 17 wherein the visual display comprises one or more multi-color LEDs, operable to generate colors according to instructions from the processor of the insulin administering device, thereby encoding said at least one message.

23. The device of claim 12 wherein said communication channel is a capacitive communication channel.

24. The device of claim 23 wherein the insulin administering device comprises a processor operable to encode said at least one message into a capacitive profile and at least one capacitive profile output mechanism operable to represent the capacitive profile.

25. The device of claim 24 wherein the capacitive profile comprises a sequence of capacitive states varying over a period of time.

26. The device of claim 24 wherein the capacitive profile comprises a plurality of regions, each exhibiting a capacitive state.

27. The device of claim 26 wherein each region of the plurality of regions exhibits a sequence of capacitive states varying over a period of time.
